(19) United States
(12) Patent Application Publication
(10) Pub. No.: US 2010/0317853 A1
(43) Pub. Date: Dec. 16, 2010

(54) NOVEL IMATINIB CAMSYLATE AND METHOD FOR PREPARING THEREOF

(76) Inventors: Yoon Seok Oh, Gyeonggi-do (KR); Jae Kyung Lim, Gyeonggi-do (KR); Dong Hyuk Shin, Gyeonggi-do (KR); Seung Kyoo Seong, Incheon (KR); Sang Ho Lee, Gyeonggi-do (KR); Hak Soo Lee, Gyeonggi-do (KR); Eun Hee Cho, Gyeonggi-do (KR); Jei Man Ryu, Gyeonggi-do (KR)

Correspondence Address: MICHAEL BEST & FRIEDRICH LLP, 100 East Wisconsin Avenue, Suite 3300, Milwaukee, WI, 53202, US

(21) Appl. No.: 12/525,962

(22) PCT Filed: Feb. 1, 2008
(86) PCT No.: PCT/KR08/00639

§ 371 Date: Aug. 25, 2009

(30) Foreign Application Priority Data
Feb. 5, 2007 (KR) 10-2007-0011556

(51) Int. Cl. C07D 401/14

(52) U.S. Cl. 544/295

(57) ABSTRACT

The present invention relates to a novel imatinib camsylate and a method for preparing the same. Imatinib camsylate according to the present invention has a faster absorption rate and higher absorption concentration in terms of pharmacokinetics, and further has excellent water solubility, as compared to commercially available imatinib mesylate.

### TECHNICAL FIELD

The present invention relates to a novel imatinib camsylate and a method for preparing the same.

### BACKGROUND ART

Imatinib is a common name of 4-[(4-methyl-1-piperazinyl)methyl]-N-[4-methyl-3-[[4-(3-pyridyl)-2-pyrimidinyl]amino]phenyl]benzamide, and is the first anticancer agent that has little effect on normal cells but effect on leukemia cells having an abnormal chromosome called the philadelphia chromosome (due to a reciprocal translocation between chromosomes 9 and 22), so as to inhibit tumor cell proliferation and promote tumor cell death.

U.S. Pat. No. 5,521,184 discloses imatinib and a preparation method thereof. For pharmaceutical use, imatinib is suitably administered as a pharmaceutically acceptable acid salt thereof. For example, imatinib is currently marketed under the brand name GLIVEC (or GLEEVEC) as monomethanesulfonate salt (imatinib mesylate) in many countries.

Subsequently, WO 2005/075454 A2 applied by Novartis discloses various pharmaceutically acceptable salt forms of imatinib, which are exemplified by a tartrate salt (D,L), a hydrochloride salt, a citrate salt, a malate salt, a D-malate salt, a fumarate salt, a succinate salt, a benzoate salt, a benzenesulfonate salt, a pamoate salt, a formate salt, a malonate salt, a 1,5-naphthalenedisulfonate salt, a salicylate salt, a cyclohexanesulfamate salt, a lactate salt, a (S)-lactate salt, a mandelate salt, an (R)-(−)-mandelate salt, a glutarate salt, an adipate salt, a squarate salt, a vanillate salt, an oxaloacetate salt, an ascorbate salt, an (L)-ascorbate salt and a sulfate salt, and discloses a preparation method thereof and their water solubility. However, there is no mention of salts being excellent in terms of various properties, in particular, pharmacokinetic properties.

Meanwhile, there has been no report on D-(+)-camsylate, L-(−)-camsylate and D,L-(±)-camsylate of imatinib, among various acid addition salts of imatinib.

Accordingly, the present inventors have prepared imatinib camsylate by using relatively low toxic 10-camphorsulphonic acid. They found that the prepared imatinib camsylate has a faster absorption rate and higher absorption concentration in terms of pharmacokinetics, and further has excellent water solubility, as compared to commercially available imatinib mesylate, thereby completing the present invention.

### DISCLOSURE OF INVENTION

### Technical Problem

It is an object of the present invention to provide a novel imatinib camsylate and a method for preparing the same.

### ADVANTAGEOUS EFFECTS

Imatinib camsylate according to the present invention has a faster absorption rate and higher absorption concentration in terms of pharmacokinetics, and further has excellent water solubility, as compared to commercially available imatinib mesylate.

### BRIEF DESCRIPTION OF THE DRAWINGS

FIG. 1 is a graph showing pharmacokinetic properties of D-(+)-camsylate, L-(−)-camsylate, and D,L-(±)-camsylate of imatinib according to the present invention.

### BEST MODE FOR CARRYING OUT THE INVENTION

The present invention provides imatinib camsylate represented by the following Formula 1:

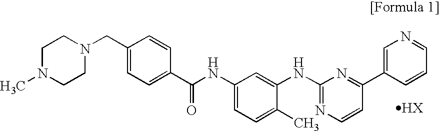

wherein HX is D-(+)-camphorsulphonic acid, L-(−)-camphorsulphonic acid or D,L-(±)-camphorsulphonic acid.

Further, the present invention provides a method for preparing imatinib camsylate of Formula 1, comprising the steps of:

1) solubilizing imatinib of the following Formula 2 in an organic solvent;

2) adding one acid selected from the following Formulae 3 and 4 or a mixture (1:1) thereof, or adding the acid or mixture solubilized in an organic solvent to the reaction solution of step 1) to prepare a mixture;

3) stirring the mixture and filtering precipitated solids to form acid addition salts; and

4) if necessary, dissolving the acid addition salts in an organic solvent to recrystallize and purify the acid addition salts.

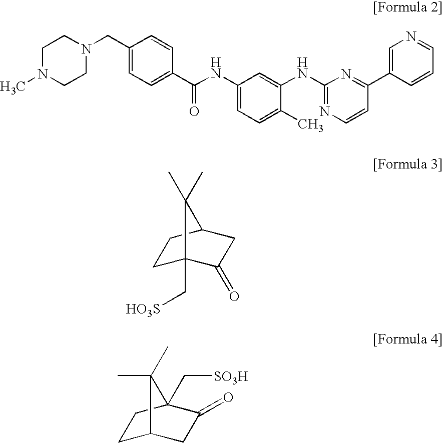

Preferably, in the preparation method, both step 3) of forming the acid addition salts and step 4) of purifying the acid addition salts may further comprise the steps of washing and drying the resulting solid after filtration.

In order to effectively promote crystallization in step 1), imatinib is preferably used in a concentration of 2 to 60% by weight, and more preferably 5 to 20% by weight, based on the total weight of the reaction solution.

In step 2), as an acid, D-(+)-10-camphorsulphonic acid of Formula 3, L-(−)-10-camphorsulphonic acid of Formula 4, or racemic D,L-(±)-10-camphorsulphonic acid, which is a mixture (1:1) of Formulae 3 and 4, is preferably used. Camphorsulphonic acid is a safe acid that is widely used in medicine, and a stable colorless solid having no moisture absorption and corrosiveness. Further, camphorsulphonic acid is harmless to human, thereby being safely and easily used for mass-production. Camphorsulphonic acid is preferably used in an amount of 0.5 to 3 molar equivalent, and more preferably 1.0 to 1.3 molar equivalent, based on 1 molar equivalent of imatinib.

Examples of the organic solvent used in steps 1), 2) and 4) may include C₁˜C₄ lower alcohol such as methanol, ethanol, isopropanol, etc.; hydrocarbons such as pentane, hexane, cyclohexane, etc.; ethers such as tetrahydrofuran, 1,4-dioxane, etc.; polar solvents such as acetone, dimethylformamide, dimethylsulfoxide, etc.; and mixtures thereof.

In steps 3) and 4), the formation and purification of acid addition salts are preferably performed in a temperature range of −10 to 120° C., and more preferably in a temperature range of 25 to 90° C.

Imatinib camsylate according to the present invention has a faster absorption rate and higher absorption concentration in terms of pharmacokinetics, and further has excellent water solubility, as compared to commercially available imatinib mesylate.

Hereinafter, the preferred Examples are provided for better understanding. However, these Examples are for the illustrative purpose only, and the invention is not intended to be limited by these Examples.

### MODE FOR THE INVENTION

### Example 1

### Preparation of Imatinib D,L-(±)-camphorsulphonic Acid Salt

5 g of 4-[(4-methyl-1-piperazinyl)methyl]-N-[4-methyl-3-[[4-(3-pyridyl)-2-pyrimidinyl]amino]phenyl]-benzamide was added to 20 ml of methanol. While stirring, 2.4 g of D,L-(±)-camphorsulphonic acid and 0.1 g of activated carbon were slowly added to the mixture, and further stirred at room temperature for 1 hr. The solution was filtered, washed with 5 ml of methanol, and then distilled off under reduced pressure. Then, 50 ml of isopropanol was added thereto, and stirred at room temperature for 1 hr. The solid mixture was filtered and washed with 10 ml of isopropanol, and then dried under reduced pressure to give 6.7 g of solid (91.1%).

Melting point (m.p.): 144˜148° C.

### Example 1-1

### Another Preparation of Imatinib D,L-(±)-camphorsulphonic Acid Salt

5 g of 4-[(4-methyl-1-piperazinyl)methyl]-N-[4-methyl-3-[[4-(3-pyridyl)-2-pyrimidinyl]amino]phenyl]-benzamide was added to 20 ml of tetrahydrofuran. While stirring, 2.4 g of D,L-(±)-camphorsulphonic acid was added to the mixture, and further stirred at room temperature for 1 hr. 10 ml of tetrahydrofuran was added to the reaction solution, and refluxed under stirring for 1 hr. Then, the solution was cooled, and filtered. The resultant was washed with 10 ml of tetrahydrofuran, and dried under reduced pressure to give 6.9 g of solid (93.8%).

Melting point (m.p.): 144˜148° C.

### Example 2

### Preparation of Imatinib D-(+)-camphorsulphonic Acid Salt

5 g of 4-[(4-methyl-1-piperazinyl)methyl]-N-[4-methyl-3-[[4-(3-pyridyl)-2-pyrimidinyl]amino]phenyl]-benzamide was added to 20 ml of methanol. While stirring, 2.4 g of D-(+)-camphorsulphonic acid and 0.1 g of activated carbon were slowly added thereto, and further stirred at room temperature for 1 hr. The solution was filtered, washed with 5 ml of methanol, and then distilled off under reduced pressure. Then, 50 ml of isopropanol was added thereto, and stirred at room temperature for 1 hr. The solid mixture was filtered and washed with 10 ml of isopropanol, and then dried under reduced pressure to give 4.8 g of solid (65.2%).

Melting point (m.p.): 130˜132° C.

### Example 3

### Preparation of Imatinib L-(−)-camphorsulphonic Acid Salt

5 g of 4-[(4-methyl-1-piperazinyl)methyl]-N-[4-methyl-3-[[4-(3-pyridyl)-2-pyrimidinyl]amino]phenyl]-benzamide was added to 20 ml of methanol. While stirring, 2.4 g of L-(−)-camphorsulphonic acid and 0.1 g of activated carbon were slowly added thereto, and further stirred at room temperature for 1 hr. The solution was filtered, washed with 5 ml of methanol, and then distilled off under reduced pressure. Then, 50 ml of isopropanol was added thereto, and stirred at room temperature for 1 hr. The solid mixture was filtered and washed with 10 ml of isopropanol, and then dried under reduced pressure to give 5.8 g of solid (78.5%).

Melting point (m.p.): 135˜136° C.

### Experimental Example 1

### Pharmacokinetic Properties of Imatinib Camsylate

In order to confirm the pharmacokinetic properties of imatinib camsylate according to the present invention, the following experiment was performed.

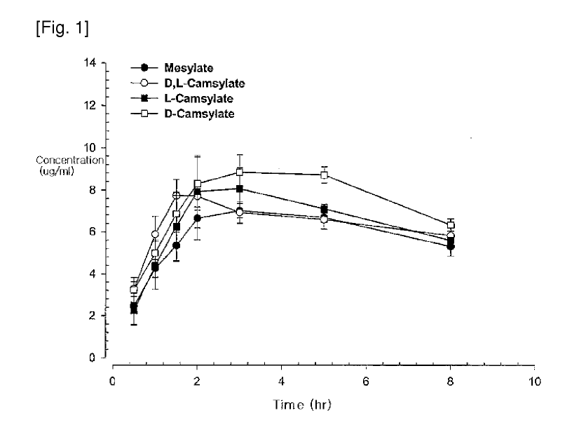

Male SD rats (body weight of 180˜220 g) were orally administered with 50 mg/kg of each imatinib camsylate prepared in Examples 1 to 3. After 0.5, 1, 1.5, 2, 2.5, 3, 5, and 8 hrs, blood samples were collected from the rats to isolate blood plasma. HPLC was performed to determine the concentration of imatinib in blood plasma. As a control group, commercially available imatinib mesylate was used. The animals used in the experiment were fasted for 16 hrs before drug administration. The concentration of imatinib according to time after drug administration is shown in Table 1 and FIG. 1.

TABLE 1

Salt of imatinib

| Time | | D,L-((±)- | L-(−)- | D-(+)- |
|---|---|---|---|---|
| (hr) | Mesylate | camsylate | camsylate | camsylate |
| 0.5 | 2.444 | 3.275 | 2.237 | 3.231 |
| 1.0 | 4.253 | 5.877 | 4.387 | 4.979 |
| 1.5 | 5.334 | 7.728 | 6.262 | 6.860 |
| 2.0 | 6.646 | 7.704 | 7.930 | 8.308 |
| 3.0 | 7.009 | 6.926 | 8.075 | 8.849 |
| 5.0 | 6.691 | 6.591 | 7.112 | 8.731 |
| 8.0 | 5.304 | 5.829 | 5.326 | 6.339 |

As shown in Table 1 and FIG. 1, it was found that imatinib camsylate according to the present invention has a faster absorption rate and higher absorption concentration in terms of pharmacokinetics than commercially available imatinib mesylate.

### Experimental Example 2

### Solubility Test

Water solubility of each imatinib camsylate prepared in Examples 1 to 3 was measured at 25° C. As a control group, commercially available imatinib mesylate was used.

The results are shown in Table 2.

TABLE 2

| Example | Salt | Solubility (mg/ml) |
|---|---|---|
| 1 | D,L-(±)-camphorsulphonic | ≧3000 |
| | acid | |
| 2 | D-(+)-camphorsulphonic | ≧3000 |
| | acid | |
| 3 | L-(−)-camphorsulphonic | ≧3000 |
| | acid | |
| Control group | methanesulphonic acid | ≧1200 |

As shown in Table 2, it was found that imatinib camsylate according to the present invention had much higher solubility than commercially available imatinib mesylate.

1. Imatinib camsylate represented by the following Formula 1: 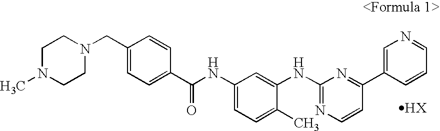
wherein HX is D-(+)-camphorsulphonic acid, L-(−)-camphorsulphonic acid, or D,L-(±)-camphorsulphonic acid.

2. The imatinib camsylate according to claim 1, wherein imatinib camsylate is D-(+)-camsylate, L-(−)-camsylate, or D,L-(±)-camsylate of imatinib.

3. A method for preparing imatinib camsylate of the following Formula 1, comprising the steps of:
1) solubilizing imatinib of the following Formula 2 in an organic solvent;
2) adding one acid selected from the following Formulae 3 and 4 or a mixture (1:1) thereof, or adding the acid or mixture solubilized in an organic solvent to the reaction solution of step 1) to prepare a mixture;
3) stirring the mixture and filtering precipitated solids to form acid addition salts; and
4) if necessary, dissolving the acid addition salts in an organic solvent to recrystallize and purify the acid addition salts. 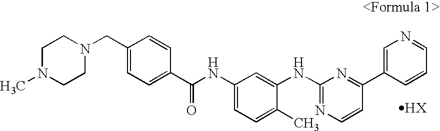
wherein HX is D-(+)-camphorsulphonic acid, L-(−)-camphorsulphonic acid, or D,L-(±)-camphorsulphonic acid.

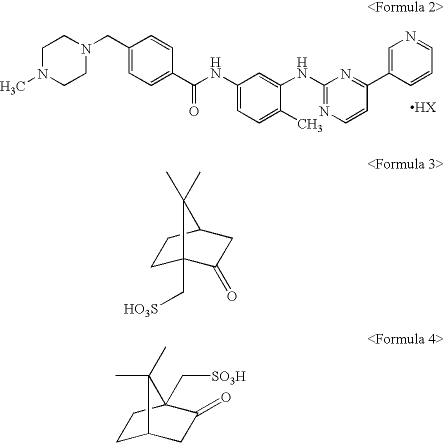

4. The method for preparing imatinib camsylate according to claim 3, wherein in step 1), imatinib is used in a concentration of 2 to 60% by weight, based on the total weight of the reaction solution.

5. The method for preparing imatinib camsylate according to claim 3, wherein in step 2), acid is used in an amount of 0.5 to 3 molar equivalent, based on 1 molar equivalent of imatinib.

6. The method for preparing imatinib camsylate according to claim 3, wherein in steps 1), 2), and 4), the organic solvent is selected from the group consisting of methanol, ethanol, isopropanol, pentane, hexane, cyclohexane, tetrahydrofuran, 1,4-dioxane, acetone, dimethylformamide, dimethylsulfoxide, and mixtures thereof.

7. The method for preparing imatinib camsylate according to claim 3, wherein in steps 3) and 4), the formation and purification of acid addition salts are performed in a temperature range of −10 to 120° C.
